(19) United States
(12) Patent Application Publication
(10) Pub. No.: US 2003/0224060 A1
(43) Pub. Date: Dec. 4, 2003

(54) Polyester polyol nanocapsules, compositions thereof, and methods of use

(75) Inventors: Jean-Thierry Simonnet, Paris (FR); Gilles Guerin, Eaubonne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/364,364

(22) Filed: Feb. 12, 2003

Related U.S. Application Data
(60) Provisional application No. 60/361,707, filed on Mar. 6, 2002.

(30) Foreign Application Priority Data
Feb. 22, 2002 (FR) 0202290

(51) Int. Cl. A61K 9/16; A61K 9/50

(52) U.S. Cl. 424/497

(57) ABSTRACT

The present invention relates generally to nanocapsules having a core-envelope structure where the core contains a lipophilic active agent and the envelope contains at least one polyester polyol obtained by polycondensation of an aliphatic dicarboxylic acid with at least two alkanediols or with at least one alkanediol and at least one hydroxyalkyl alkanediol. These nanocapsules make it possible to stabilize lipophilic active agents that are difficult to stabilize, especially retinol esters such as retinyl propionate.

### REFERENCE TO PRIOR APPLICATIONS

This application is based on, and claims priority to, U.S. provisional application 60/361,707, filed Mar. 6, 2002 and French patent application 0202290, filed Feb. 22, 2002, both of which are incorporated by reference herein.

### SUMMARY OF THE INVENTION

The present invention relates to nanocapsules having a lipid core forming or containing a lipophilic active agent, and a water-insoluble envelope, comprising at least one polyester polyol. Preferably, this polyester polyol is obtained by polycondensation of an aliphatic dicarboxylic acid with at least two alkanediols or with at least one alkanediol and at least one hydroxyalkyl alkanediol. The aliphatic dicarboxylic acid is preferably adipic acid (hexane-1,6-dioic acid). These nanocapsules make it possible to stabilize lipophilic active agents that are difficult to stabilize, and especially retinol esters such as retinyl propionate. The invention also relates to cosmetic and/or dermatological compositions containing the invention nanocapsules, and to a process for preparing these nanocapsules.

### BACKGROUND OF THE INVENTION

The term “nanoparticles” mainly encompasses two different systems: “nanospheres” consisting of a porous polymer matrix in which the active principle is absorbed and/or adsorbed, and “nanocapsules” with a structure of core-envelope type, i.e. a structure with a lipid core forming or comprising the active principle, this core being encapsulated in a water-insoluble, preferably continuous, protective envelope. The present invention relates exclusively to this second vesicular type of nanoparticles, i.e. nanocapsules with a lipid core surrounded by a polymer membrane.

The encapsulation or absorption of lipophilic active principles in particles of submicron dimensions (less than 1 μm) has been known for several years and is widely used in particular in the fields of cosmetology and dermatology. Specifically, these particles, known as nanoparticles, are capable of crossing the superficial layers of the stratum corneum and of penetrating into the upper layers of the living epidermis to release the active principle therein. This penetration into deeper layers broadens the space of action of the active principles and shelters them from rapid elimination by simple rubbing.

The encapsulation of active principles in capsules of submicron size does admittedly make it possible to transport active molecules more deeply into the skin, but it does not always ensure sufficient stability of the active agent with respect to the surrounding physicochemical conditions, especially for active agents that are sensitive to the environment (light, oxygen or heat) and especially sensitive to oxidation and to hydrolysis. The problem of the instability of active agents that are sensitive to oxidation and to hydrolysis in aqueous medium arises in particular when it is desired to introduce them into a medium containing water and/or to handle them in the open air. Now, for various reasons associated in particular with better user comfort (softness, emollience and the like), cosmetic or dermatological compositions, especially skincare compositions, are usually in the form of an emulsion consisting of an aqueous phase and an oily phase, either in the form of an oil-in-water (O/W) emulsion or in the form of a water-in-oil (W/O) emulsion. The lipophilic active agents are generally introduced into the oily phase of the emulsion, but, on account of the presence of an aqueous phase, they have a tendency to be destabilized and thus to lose their activity, which runs counter to the desired effect.

Examples of lipophilic active agents that have a tendency to be destabilized in aqueous medium, which may be mentioned, include retinol and its derivatives and especially its esters, more particularly the short-chain esters, the esters having all the more tendency to be hydrolysed the shorter the alkyl chain (C2 to C10).

One solution for stabilizing retinol consists in encapsulating it in poly(alkylene adipate)-based nanocapsules, as described in document FR-A-2 787 730, the term poly(alkylene adipate) encompassing homopolymers of adipic acid and of an alkanediol and copolymers of poly(ester ether) type, obtained from adiopic acid and from one or more alkanediols and/or ether-diols and/or triols.

However, only polymers obtained from adipic acid and one alkanediol and having a molar mass of 10 000 were exemplified in this document, and it has been observed that these nanocapsules do not make it possible to stabilize retinol esters sufficiently, especially those that are sensitive to hydrolysis in aqueous medium, i.e. those with a short alkyl chain such as, for example, retinyl propionate.

There is thus still a need for a material for obtaining good stability of active agents that are sensitive to an oxidizing and/or aqueous environment, such as retinol and its derivatives, especially its hydrolysis-sensitive esters.

### DETAILED DESCRIPTION OF THE INVENTION

The present invention satisfies the above-stated need. Specifically, the inventors have discovered that encapsulation in nanocapsules based on a particular type of polyester polyol gives a spectacular improvement in the stability of lipophilic active agents that are sensitive to oxidation and to hydrolysis, especially retinol esters.

Thus, as will be seen in the examples hereinbelow, and representative of the invention as a whole, the encapsulation of retinyl propionate in nanocapsules according to the invention gives this active molecule superior stability. (For example, in Example 3 a loss of activity of only 12 or 15% after one month of storage at 45° C. is observed with the invention, whereas, under equivalent conditions, this same molecule encapsulated in polymers of the prior art [poly(ethylene adipate) PEA] shows a loss of activity of 31%).

A subject of the invention is thus a nanocapsule comprising, consisting of, and consisting essentially of:

a lipid core comprising, consisting of, and consisting essentially of at least one lipophilic active agent, and

a water-insoluble, preferably continuous, polymer envelope, comprising, consisting, and consisting essentially of at least one polyester polyol, this polyester polyol preferably being obtained by polycondensation of at least one aliphatic dicarboxylic acid with at least two alkanediols or with at least one alkanediol and at least one hydroxyalkyl alkanediol optionally comprising an alkyl chain, wherein the polyester polyol preferably has a weight-average molar mass ( molecular weight) less than 5000.

The nanocapsules according to the invention give excellent stability results for sensitive active agents, especially for C1-C6 retinol esters, which are highly hydrolysis- sensitive molecules, such as retinyl acetate and retinyl propionate.

The polyester polyols that may be used to form the envelope of the nanocapsules are not limited, and preferably are copolymers obtained by polycondensation of at least one aliphatic dicarboxylic acid with at least two alkanediols, or with at least one alkanediol and at least one hydroxyalkyl alkanediol, and optionally a small proportion of triols. Mixtures may be used.

The aliphatic dicarboxylic acid used for the preparation of the polyester polyols is not limited. Mixtures may be used. Preferred aliphatic dicarboxylic acids include, for example, malonic acid, succinic acid, glutaric acid, adipic acid (or hexane-1,6-dioic acid), pinelic acid, sebacic acid and azelaic acid, and mixtures thereof. According to one preferred embodiment of the invention, the dicarboxylic acid is adipic acid.

The alkanediols used for the preparation of the polyester polyols are not limited. Mixtures may be used. Preferred alkanediols include alkanediols with a linear or branched chain containing from 2 to 20 carbon atoms and preferably from 2 to 10 carbon atoms. They may be chosen especially from ethylene glycol, propylene glycol, 1,3-propanediol, 1,4-butanediol, 1,5-pentanediol, 1,6-hexanediol and neopentyl glycol, and mixtures thereof. According to one preferred embodiment of the invention, the alkanediol is selected from the group consisting of 1,4-butanediol and 1,6-hexanediol, and mixtures thereof. More preferably, the alkanediol is 1,4-butanediol.

In the present patent application, the expression “hydroxyalkyl alkanediols optionally comprising an alkyl chain” means alkanediols comprising at least one hydroxyalkyl group and also possibly comprising an alkyl chain, in which the hydroxyalkyl group and the alkyl chain are, independently of each other, linear or branched, saturated chains containing from 1 to 10 carbon atoms. The hydroxyalkyl alkanediols that may be used to form the polyester polyols of the present invention are not limited, and mixtures may be used. Useful hydroxyalkyl alkanediols optionally comprising an alkyl chain include, for example, 2-alkyl-2-(hydroxyalkyl)-1,3-propanediol, in which the hydroxyalkyl group and the alkyl chain contain, independently of each other, from 1 to 10 carbon atoms, such as, for example, 2-ethyl-2-(hydroxymethyl)-1,3-propanediol and 2-methyl-2-(hydromethyl)-1,3-propanediol; 2-(hydroxyalkyl)-1,3-propanediol, in which the hydroxyalkyl group contains from 1 to 10 carbon atoms; and mixtures thereof.

According to one preferred embodiment of the invention, the hydroxyalkyl alkanediol used is 2-ethyl-2-(hydroxymethyl)-1,3-propanediol.

As indicated above, the polyester polyols useful in preparing the nanocapsules of the invention may also contain a limited number of triol-based branching units. The triols used are not limited and are generally selected from the group consisting of glycerol, trimethylolethane and trimethylolpropane. The fraction of branching units derived from the above triols preferably generally does not exceed 5 mol % relative to all of the units derived from diols and triols.

According to one preferred embodiment of the present invention, the polyester polyol forming the envelope of the nanocapsules is selected from the group consisting of polyester polyols obtained from adipic acid, from 1,4-butanediol and from 1,6-hexanediol and from polyester polyols obtained from adipic acid, from 1,4-butanediol and from 2-ethyl-2-(hydroxymethyl)-1,3-propanediol. Examples of preferred polyester polyols that may be mentioned include those sold by the company Inolex under the names Lexorez and especially Lexorez 1151-35 and Lexorez 1460-36. Polyester polyols obtained obtained from adipic acid, from 1,4-butanediol and from 2-ethyl-2-(hydroxymethyl)-1,3-propanediol are particularly preferred.

The polyester polyols used in the present invention preferably have a weight-average molar mass (molecular weight) (measured by gel permeation chromatography) of less than 5000 including all values and subranges within this limitation (e.g., 4500, 4000, 3500, 3000, 2500, etc.), and preferably less than 4000, including for example from 1000 to 4500, from 1000 to 4000, and from 2000 to 4000. Their viscosity at 60° C. is preferably less than 6 000 cPoises (=6 Pa.s); including all values and subranges within this limitation (e.g., 5500, 5000, 4500, 4000, 3500, 3000, 2500, 2000, etc.). The polyester polyol Lexorez 1151-35 has at 60° C. a viscosity of 3000 cps and the polyester polyol Lexorez 1460-36 has at 60° C. a viscosity of 3500 cps.

The polyester polyols used in the present invention preferably have a melting point of from 30 to 90° C., including all values and subranges therebetween, including for example 35 to 70° C.

The polyester polyols used in the present invention may be prepared according to processes usually used for preparing polyesters.

The polyester polyols described above are used to prepare nanocapsules comprising, consisting of, and consisting essentially of a lipid core, the lipid core comprising, consisting of, and consisting essentially of a lipophilic active agent, the lipid core being surrounded by an envelope formed from these polyester polyol polymers.

The general process for preparing nanocapsules which is preferred herein is one described in another context in EP-A-0 274 961, comprising:

in dissolving the polymer, the lipid phase forming or containing the active agent and optionally a coating agent in a suitable organic solvent, i.e. a solvent that is water-miscible, non-toxic and more volatile than water (generally acetone and/or a lower alcohol),

in preparing a solution of a suitable surfactant in water (non-solvent for the polymer and for the lipid phase),

in mixing the organic phase and the aqueous phase together in any order (mixing) preferably adding the organic phase to the aqueous phase, while stirring moderately, which results in the spontaneous formation of a nanocapsule emulsion,

and then in evaporating the organic phase and, optionally, some of the aqueous phase (for example at a temperature of 35 to 40° C.), to obtain a concentrated suspension of nanocapsules in an aqueous phase.

Thus, a subject of the invention is a process for preparing the nanocapsules according to the invention, comprising:

dissolving an invention polyester polyol polymer, component(s) of the eventual lipid phase (e.g., at least one active agent), and optionally a coating agent, in a water-miscible organic solvent, to prepare an organic phase,

mixing the organic phase with an aqueous phase comprising surfactant, preferably by adding the organic phase to an aqueous phase comprising surfactant, while stirring, preferably moderately,

and then evaporating the organic phase and, optionally, some of the aqueous phase.

This preparation process generally involves heating the organic phase and/or the aqueous phase, e.g., to temperatures of between 35 and 70° C. The polyester polyols used in the present invention make it possible to perform this process at room temperature, which is an important advantage in particular for heat-sensitive active substances such as retinol.

When the active agent is sensitive to oxidation and/or to heat, the nanocapsules are preferably prepared under inactinic light, under an inert atmosphere and at room temperature.

While not being bound by a particular theory, the surfactant dissolved in the aqueous phase (aqueous surfactant solution) used during the preparation of the nanocapsules is believed to serve mainly to control the size of the nanocapsules. The reason for this is that it ensures the stability of the nanocapsules in the emulsion resulting from the addition of the organic (e.g.,acetone) phase to the aqueous phase, and prevents them from coalescing. Any surfactant of hydrophilic nature, whether nonionic, anionic or cationic, can be used. Mention may be made, for example, of sodium lauryl sulphate, quaternary ammonium compounds, polyoxyethylenated or non-polyoxyethylenated sorbitan monoesters, fatty alkyl ethers of polyoxyethylene glycol, the condensates of ethylene oxide and of propylene oxide, such as the products Pluronic® F-68 or Pluronic® F-108 sold by the company BASF, or phospholipids such as lecithin. The weight ratio of the surfactant to the materials constituting the nanocapsules is advantageously between 0.01 and 0.5 and preferably in the region of 0.2.

The nanocapsules of the invention may optionally be provided with a so-called “lamellar” coating. This is a structure organized as one or more lipid leaflet(s) each consisting of a bilayer of amphiphilic molecules which is similar to that of biological membranes. The polymer envelope of the nanocapsules according to the invention may thus be surrounded with a lamellar coating whose structure is organized as one or more leaflet(s) each consisting of a double layer of amphiphilic molecules constituting a coating agent. Besides its function of adjusting the size of the nanocapsules, this coating improves the leaktightness of the nanocapsules with respect to a leakage of the active principle into another lipid phase of the composition.

The coating agents are surfactants of hydrophobic nature, which are soluble in the organic phase used in the above-described process and which are capable, in the presence of water, of forming the lipid double layers described above. In the process for encapsulating active principles of the invention this coating agent is dissolved in the organic (e.g., acetonic/alcoholic) phase containing the polymer and the lipid phase.

Examples of such coating agents include phospholipids such as lecithin according to patent application EP-A-447,318; certain polycondensates of ethylene oxide and of propylene oxide, such as the products sold under the name Pluronic® by the company BASF, such as Pluronic® L121 or under the name Synperonic® by the company ICI; or silicone surfactants (silicones comprising at least one oxyethylenated and/or oxypropylenated chain) capable of forming lamellar structures, such as those described in documents U.S. Pat. No. 5,364,633 and U.S. Pat. No. 5,411,744 and used in patent application FR-A-2,742,677, for example those sold by the company Dow Corning under the names DC 5329, DE 7439-146, DC 2-5695 and Q4-3667; and mixtures thereof.

The average size of the nanocapsules based on polyester polyols according to the invention is preferably generally less than 1 micron and preferably less than 500 nm, all values and subranges within these limits being expressly included. It is advantageously between 50 and 800 nm, preferably between 100 and 400 nm. This size is determined, for example, using a laser granulometer (Amtech BI 90 model from the company Brookhaven Instrument).

The nanocapsules of the present invention may contain active agents of any kind, such as cosmetic or dermatological lipophilic active agents. Examples include vitamins and derivatives thereof, emollients, anti-inflammatory agents, antibacterial agents, antifungal agents, antiviral agents, anti-seborrhoeic agents, anti-acne agents, keratolytic agents, antihistamine agents, anaesthetics, cicatrizing agents, pigmentation modifiers, sunscreens, free-radical traps and moisturizers, and mixtures thereof.

According to the present invention, the encapsulated lipophilic active agent is preferably selected from the group consisting of lipophilic active agents that are sensitive to the surrounding physicochemical conditions such as the temperature, the pH, light or the presence of oxidizing agents, and in particular active agents that are sensitive to oxidation and to hydrolysis.

Examples of preferred lipophilic active agents include retinol (vitamin A) and its esters, in particular the esters containing from 1 to 30 carbon atoms and more especially the esters containing from 1 to 10 carbon atoms, especially those containing from 1 to 6 carbon atoms, which are more sensitive. Mention may especially be made of retinyl propionate, retinyl acetate, retinyl butyrate, retinyl pivalate, retinyl valerate, retinyl hexanoate, retinyl heptanoate, retinyl cyclopentanecarboxylate, retinyl caprate and retinyl caprylate; and mixtures thereof.

Lipophilic active agents that may also be mentioned include vitamin E or its esters such as tocopheryl acetate; vitamin D or its derivatives; vitamin F or its derivatives; carotenes such as β-carotene and its derivatives such as lycopene; salicylic acid or its derivatives, especially those described in documents FR-A-2 581 542, EP-A-378 936 and EP-A-570 230, in particular 5-n-octanoylsalicylic acid, 5-n-decanoylsalicylic acid, 5-n-dodecanoylsalicylic acid, 5-n-octylsalicylic acid, 5-n-heptyloxysalicylic acid and 4-n-heptyloxysalicylic acid; steroids such as dehydroepiandrosterone (or DHEA), and also (1) its biological derivatives and precursors, in particular DHEA salts and esters, such as DHEA sulphate and salicylate, 7-hydroxy DHEA, 7-ceto DHEA, 7-hydroxy esters of 7-ceto DHEA, especially 3-beta-acetoxy-7-oxo DHEA, and (2) its chemical derivatives and precursors, in particular sapogenins such as diosgenin or hecogenin, and/or derivatives thereof such as hecogenin acetate, and/or natural extracts containing them and especially extracts of dioscorea plants, such as wild yam; and mixtures thereof.

The nanocapsules according to the present invention may exist in an aqueous suspension, as shown by the preparation process described above. Thus, the invention also relates to an aqueous suspension of nanocapsules containing, in an aqueous medium, nanocapsules based on polyester polyols as described above.

The nanocapsules according to the invention and the aqueous suspensions containing them may be used especially in compositions for topical application, in particular cosmetic and/or dermatological compositions.

A subject of the invention is thus a composition for topical application comprising a physiologically acceptable medium, one or more nanocapsules as described above, or an aqueous suspension of nanocapsules. The composition may especially constitute a cosmetic or dermatological composition.

The expression “physiologically acceptable medium” means a medium that is compatible with keratin materials such as the skin, mucous membranes, the nails, the scalp and the hair.

The amount of nanocapsules in the compositions of the present invention intended for topical application preferably generally ranges from 0.1% to 30% by weight and preferably from 0.5% to 15% by weight, relative to the total weight of the composition.

The compositions according to the invention may be in any form. They may be, for example, in the form of a serum, a lotion, an aqueous, aqueous-alcoholic or oily gel, a water-in-oil or oil-in-water or multiple (W/O/W or O/W/O) emulsion, or alternatively in the form of an aqueous dispersion of lipid vesicles consisting of ionic or nonionic lipids or of a mixture thereof, these vesicles optionally containing an oily phase.

In addition, the compositions used according to the invention may be more or less fluid and may have the appearance of a white or coloured cream, a pomade, a milk, a lotion, a serum, a paste or a mousse. They may optionally be applied to the skin in aerosol form. They may also be in solid form, and for example in cast form, as a dish, or in the form of a stick.

The physiologically acceptable medium may comprise an aqueous phase and/or an oily phase. It preferably generally comprises at least one aqueous phase since the nanocapsules are usually in an aqueous suspension. This aqueous phase of the compositions according to the invention comprises at least water. Depending on the pharmaceutical form of the composition, the amount of aqueous phase preferably generally ranges from 40% to 100% by weight relative to the total weight of the composition, and more preferably from 60% to 95% by weight relative to the total weight of the composition. The amount of water may represent all or some of the aqueous phase, and it is preferably generally at least 30% by weight relative to the total weight of the composition.

The compositions of the invention may comprise, in the aqueous phase and/or in the oily phase if it comprises an oily phase, one or more hydrophilic, lipophilic and/or amphiphilic organic solvents that are physiologically acceptable, i.e. well tolerated and giving a cosmetically acceptable feel.

The organic solvents may represent for example from 0.5% to 50% and preferably from 2% to 20% of the total weight of the composition. The organic solvents may be selected from the group consisting of hydrophilic organic solvents, lipophilic organic solvents and amphiphilic solvents, or mixtures thereof.

Useful organic solvents include linear or branched lower monoalcohols containing from 1 to 8 carbon atoms, for instance ethanol, propanol, butanol, isopropanol or isobutanol; polyols such as propylene glycol, isoprene glycol, butylene glycol, propylene glycol, glycerol or sorbitol; monoalkyl or dialkyl isosorbide in which the alkyl groups contain from 1 to 5 carbon atoms, for instance dimethyl isosorbide; polyethylene glycols, especially those containing from 6 to 80 ethylene oxides, such as polyethylene glycol 32 OE; ethylene glycol ethers, for instance diethylene glycol monomethyl or monoethyl ether; propylene glycol ethers, for instance dipropylene glycol methyl ether; polyol esters and ethers, such as polypropylene glycol (PPG) esters and more especially fatty acid esters of polypropylene glycol (PPG), fatty alkyl ethers of PPG, for instance PPG-23 oleyl ether and PPG-36 oleate; fatty acid alkyl esters, such as diisopropyl adipate, dioctyl adipate or alkyl benzoates; and mixtures thereof.

When the composition comprises an oily phase, the nature of this oily phase is not critical. The oily phase may thus comprise any fatty substance, and in particular oils, for example those used in cosmetics or dermatology. The oily phase generally comprises at least one oil.

Examples of oils that may be used in the composition of the invention include:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, for instance those sold by the company Stearineries Dubois or those sold under the names Miglyol 810, 812 and 818 by the company Dynamit Nobel, jojoba oil or karite butter oil;

synthetic esters and synthetic ethersi especially of fatty acids, for instance oils of formulae R¹COOR² and R¹OR² in which R¹ represents the fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, isocetyl palmitate; 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates such as stearyl heptanoate; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as Parleam® oil;

natural or synthetic essential oils such as, for example, eucalyptus oil, lavandin oil, lavender oil, vetiver oil, Litsea cubeba oil, lemon oil, sandalwood oil, rosemary oil, camomile oil, savoury oil, nutmeg oil, cinnamon oil, hyssop oil, caraway oil, orange oil, geraniol oil, cade oil and bergamot oil;

fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or partially silicone-based fluoro oils, for instance those described in document JP-A-2 295 912;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendant or at the end of a silicone chain, these groups containing from 2 to 24 carbon atoms; phenylsilicones, for instance phenyltrimethicones, phenyldimethicones, phenyltri-methylsiloxydiphenylsiloxanes, diphenyldimethicones, diphenylmethyldiphenyltrisiloxanes, 2-phenylethyltrimethylsiloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

Other fatty substances that may be present in the oily phase are, for example, fatty acids containing from 8 to 30 carbon atoms, for instance stearic acid, lauric acid, palmitic acid and oleic acid; waxes, for instance lanolin, beeswax, camauba wax or candelilla wax, paraffin waxes, lignite wax or microcrystalline waxes, ceresin or ozokerite, synthetic waxes such as polyethylene waxes, Fischer-Tropsch waxes; petroleum jelly; gums such as silicone gums (dimethiconol); silicone resins such as trifluoromethyl-C1-4-alkyldimethicone and trifluoropropyldimethicone.

These fatty substances may be chosen in a varied manner by a person skilled in the art so as to prepare a composition having the desired properties, for example in terms of consistency or texture, in view of this disclosure.

These organic solvents, oils, fatty substances, etc., can also be present in the lipid core of the nanocapsule.

When the composition is in the form of an emulsion, the proportion of oily phase of the emulsion is not limited and may range, for example, from 5% to 80% by weight and preferably from 5% to 50% by weight relative to the total weight of the composition. The oils, emulsifiers and co-emulsifiers used in the composition in emulsion form are selected from the group consisting of those conventionally used in cosmetics or dermatology. The emulsifier and the co-emulsifier are generally present in the composition in a proportion ranging from 0.3% to 30% by weight and preferably from 0.5% to 20% by weight, relative to the total weight of the composition. The emulsion may also contain lipid vesicles.

The emulsions generally contain at least one emulsifier selected from the group consisting of amphoteric, anionic, cationic and nonionic emulsifiers, used alone or as a mixture. The emulsifiers are chosen in a suitable manner depending on the emulsion to be obtained (W/O or O/W).

Examples of emulsifiers that may be used in W/O emulsions include dimethicone copolyols such as the mixture of cyclomethicone and of dimethicone copolyol, sold under the name “DC 5225 C” by the company Dow Corning, and alkyldimethicone copolyols, such as the laurylmethicone copolyol sold under the name “Dow Corning 5200 Formulation Aid” by the company Dow Corning, the cetyldimethicone copolyol sold under the name Abil EM 90® by the company Goldschmidt, or the mixture of cetyldimethicone copolyol, polyglyceryl-4 isostearate and hexyl laurate, sold under the name Abil WE09® by the company Goldschmidt. One or more co-emulsifiers may also be added thereto, which may be advantageously selected from the group consisting of the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

Examples of emulsifiers that may be used in O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; the sugar ethers of C₈-C₂₄ fatty alcohols, and mixtures thereof.

Glyceryl esters of fatty acids that may especially be mentioned include glyceryl stearate (glyceryl mono-, di- and/or tristearate); glyceryl ricinoleate; diglyceryl monostearate; and mixtures thereof.

Polyethylene glycol esters of fatty acids that may especially be mentioned include polyethylene glycol stearate (polyethylene glycol mono-, di- and/or tristearate) and more especially polyethylene glycol 20 OE monostearate (CTFA name: PEG-20 stearate), and mixtures thereof.

Useful fatty acid esters of glucose or of alkylglucose include glucose palmitate, alkylglucose sesquistearates, for instance methyl glucose sesquistearate, alkylglucose palmitates, for instance methylglucose palmitate or ethylglucose palmitate, fatty esters of methylglucoside and more especially the diester of methylglucoside and of oleic acid (CTFA name: Methyl glucose dioleate); the mixed ester of methylglucoside and of the oleic acid/hydroxystearic acid mixture (CTFA name: Methyl glucose dioleate/hydroxysterate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and of isostearic acid (CTFA name: Methyl glucose sesquiisostearate); the mixture of the monoester and diester of methylglucoside and of stearic acid (CTFA name: Methyl glucose sesquistearate) and in particular the product sold under the name Glucate SS by the company Amerchol, and mixtures thereof.

Examples of oxyethylenated ethers of a fatty acid and of glucose or of alkylglucose include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters include sucrose palmitostearate, sucrose stearate and sucrose monolaurate.

Sugar ethers that may especially be mentioned are alkylpolyglucosides, for example decylglucoside, for instance the product sold under the name Mydol 10 by the company Kao Chemicals, the product sold under the name Plantaren 2000 by the company Henkel, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Henkel; cocoglucoside, for instance the product sold under the name Plantacare 818/UP by the company Henkel; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Henkel, and also arachidyl glucoside, for example in the form of the mixture of arachidyl alcohol and behenyl alcohol and arachidyl glucoside, sold under the name Montanov 202 by the company SEPPIC, and mixtures thereof.

The composition may also contain other surfactants, and especially anionic surfactants such as, for example, monosodium and disodium acylglutamates, for instance the disodium salt of N-stearoyl-L-glutamic acid sold under the name Acylglutamate HS21 by the company Ajinomoto.

The nanoparticles, aqueous suspension thereof and compositions thereof may contain one or more adjuvants, for example adjuvants that are common in cosmetics and dermatology, chosen, for example, from gelling agents and/or thickeners (hydrophilic or lipophilic); emollients; active agents (hydrophilic or lipophilic) other than or identical to those indicated above; free-radical scavengers; sequestering agents; antioxidants; preserving agents; acidifying or basifying agents; fragrances; film-forming agents; dyestuffs (pigments such as iron oxides and titanium dioxide, nacres and soluble dyes); fillers (for example polyethylene powder or Nylon powder); and mixtures thereof The amounts of these various adjuvants are those conventionally used in the fields under consideration, generally from 0.001% to 30% by weight and preferably from 0.1% to 20% by weight, relative to the total weight of the composition.

As examples of active agents that may be used in the invention, mention may be made of moisturizers such as protein hydrolysates and polyols, for instance glycerol, glycols, for instance polyethylene glycols, and sugar derivatives; natural extracts; anti-inflammatory agents; procyannidol oligomers; vitamins, for instance vitamin C (ascorbic acid), vitamin B5 (panthenol), vitamin B3 (niacinamide), derivatives of these vitamins (especially esters) and mixtures thereof; urea; caffeine; depigmenting agents such as kojic acid, hydroquinone and cafeic acid; α-hydroxy acids such as lactic acid and glycolic acid, and derivatives thereof; sunscreens; hydrocortisone; melatonin; algal, fungal, plant, yeast and bacterial extracts; enzymes; steroids; antibacterial active agents, for instance 2,4,4′-trichloro-2′-hydroxydiphenyl ether (or Triclosan), 3,4,4′-trichlorocarbanilide (or Triclocarban); β-hydroxy acids, for instance salicylic acid and its derivatives; matting agents, for instance fibres; tensioning agents; organic or mineral powders, especially those from 20 nm to 20 μm in size; and mixtures thereof.

Depending on the fluidity of the composition that it is desired to obtain, one or more hydrophilic or lipophilic gelling agents may be added thereto. Examples of hydrophilic gelling agents that may be mentioned include carboxyvinyl polymers such as carbomers; polyacrylamides and polymers and copolymers of 2-acrylamido-2-methylpropanesulphonic acid), optionally crosslinked and/or neutralized, for instance the poly(2-acrylamido-2-methylpropanesulphonic acid) sold by the company Hoechst under the tradename “Hostacerin AMPS” (CTFA name: Ammonium polyacryldimethyltauramide).

Useful lipophilic gelling agents include modified clays such as bentones, such as the mixture “cyclomethicone, Quaternium-18 hectorite, SD alcohol 40” (10/85/5) (CTFA name) sold under the name Bentone Gel VS-5 by the company Rheox; crosslinked elastomeric polyorganosiloxanes such as those sold under the names KSG6 from Shin-Etsu, Trefil E-505C or Trefil E-506C from Dow Corning, Gransil from Grant Industries (SR-CYC, SR DMF10 or SR-DC556), or those sold in the form of gels: KSG15, KSG17, KSG16 and KSG18 from Shin-Etsu, Gransil SR 5CYC gel, Gransil SR DMF 10 gel, Gransil SR DC 556 gel and SF 1204 and JK 113 from General Electric.

When they are present, these gelling agents are generally used at concentrations ranging from 0.01% to 10% and preferably from 0.05% to 5% by weight of active material, relative to the total weight of the composition.

Needless to say, a person skilled in the art will take care to select this or these optional additional compound(s) and the amount thereof.

The composition containing nanocapsules preferably has a pH that respects the skin and that generally ranges from 3 to 8 and preferably from 4.5 to 7.

The compositions according to the invention may be used for any suitable application depending on the encapsulated active agent.

Thus, the compositions of the invention may be used for example as care (or treating), protective, cleansing, makeup-removing and/or makeup products for keratin materials (skin, hair, scalp, eyelashes, eyebrows, nails or mucous membranes), such as protective, treating or care creams for the face, the hands or the body, protective or care body milks, and care gels or care mousses for the skin and/or mucous membranes (lips).

The compositions of the invention containing sunscreens may also be used as antisun products.

The compositions of the invention may also be used as makeup products, especially for making up the skin, the eyebrows, the eyelashes and the lips. The makeup products are usually coloured and generally contain pigments. In the form of makeup products, the compositions of the invention may advantageously constitute a foundation, a lipstick, a makeup rouge, an eyeshadow, a mascara or an eyeliner.

The compositions according to the invention may also be used as rinse-off products or as leave-on products for cleansing facial and/or body skin and/or for cleansing the hair, for example as hair products, including for caring for and conditioning the hair.

A subject of the invention is also the cosmetic use of a cosmetic composition as defined above, as a care, cleansing and/or makeup-removing product for the skin, the hair, the scalp, the eyelashes, the eyebrows, the nails or mucous membranes.

A subject of the invention is also the cosmetic use of a cosmetic composition as defined above, as a makeup product.

A subject of the invention is also the cosmetic use of a composition as defined above, as an antisun product (for protection against sunlight and/or the UV rays of tanning machines).

A subject of the invention is also the use of a composition as defined above, as a rinse-off or leave-on hair product.

Another subject of the invention is a (non-therapeutic) process for treating a keratin material (skin, scalp, hair, eyelashes, eyebrows, nails or mucous membranes), wherein a composition as defined above is applied to the keratin material. The keratin material is especially the skin.

The examples given below, purely as non-limiting illustrations, will allow the invention to be understood more clearly. The amounts therein are given as percentages by weight, except where otherwise mentioned.

### EXAMPLE 1

Preparation of Retinyl Propionate Nanocapsules

The following are dissolved, under an inert atmosphere, at room temperature and with stirring, in 100 ml of acetone in a 250 ml amber-glass round-bottomed flask:

1 g of the polyester polyol based on adipic acid, butanediol and 2-ethyl-2-(hydroxymethyl)-1,3-propanediol, sold under the name Lexorez 1151-35 by the company Inolex, and

0.5 g of DC2-5695 (oxyethylenated polydimethylsiloxane) sold by the company Dow Corning.

2.5 g of retinyl propionate are added to the solution obtained.

Separately, 0.25 g of Pluronic F108 (or Poloxamer 338) (128 OE/54 OP/128 OE) sold by the company BASF is dissolved in 150 g of water in a 500 ml amber-glass round-bottomed flask, under an inert atmosphere and at room temperature, with stirring.

The acetone phase is poured into the aqueous phase with continued stirring. Next, the mixture is evaporated in a rotary evaporator until a final volume of 50 ml is obtained.

This aqueous suspension contains (coated) nanocapsules with a mean diameter of 263 nm.

### EXAMPLE 2

Preparation of Retinyl Propionate Nanocapsules

The process is performed as described in Example 1, but replacing the polyester polyol based on adipic acid, butanediol and 2-ethyl-2-(hydroxymethyl)-1,3-propanediol with an identical weight amount of polyester polyol based on adipic acid, butanediol and hexanediol, sold under the name Lexorez 1460-36 by the company Inolex.

An aqueous suspension of (coated) nanocapsules with a mean diameter of 251 nm is obtained.

### EXAMPLE 3

Tests of Stability of the Retinol Encapsulated in Different Polymers

The stability of the retinyl propionate enclosed in the nanocapsules according to Examples 1 and 2 is compared with the stability of this active agent in nanocapsules based on poly(ethylene) adipate (referred to hereinbelow as PEA) with a molecular weight of 10 000, sold by the company Scientific Polymer Product, and based on Capa 656 (polycaprolactone with an MW of 56 000 and sold by the company Solvay), all these nanocapsules being coated with the silicone agent DC-5695.

The nanocapsules containing the retinyl propionate are stored in the form of an aqueous suspension for one month at 45° C. in closed, lightproof and gastight packaging. After this storage period, the loss of active principle (retinyl propionate) is evaluated by HPLC.

The results obtained are collated in the following table:

| | | | Lexorez | Lexorez |
|---|---|---|---|---|
| Polymer | PEA | Capa 656 | 1151-35(1) | 1460-36(2) |
| Molar mass | 10 000 | 50 000 | 3 200 | 3 200 |
| Melting point in ° C. | 55 | 58-60 | 55-65 | 40-50 |
| Coating agent | Silicone DC- | Silicone DC- | Silicone DC- | Silicone DC- |
| | 5695 | 5695 | 5695 | 5695 |
| Diameter of the capsules | 210 nm | 269 nm | 263 nm | 251 mm |
| capsules | | | | |
| pH of the | 7.0 | 8.2 | 8.1 | 7.9 |
| suspension | | | | |
| % loss after 1 month at 45° C. | 31% | 26% | 15% | 12% |

(1) Lexorez 1151-35: butanediol/2-ethyl-2-(hydroxymethyl)-1,3-propanediol/adipic acid
(2) Lexorez 1460-36: butanediol/hexanediol/adipic acid

These results show that the use of polyester polyols such as Lexorez, which are obtained from two alkanediols or from one alkanediol and one hydroxyalkyl alkanediol and which have a weight average molar mass (molecular weight) less than 5 000, improve the stability of the encapsulated vitamin A propionate.

### EXAMPLE 4

O/W Emulsion

| | |
|---|---|
| Oily phase: | |
| Diglyceryl monostearate | 2.0% |
| PEG-20 stearate | 1.5% |
| Disodium N-stearoyl-L-glutamic acid | |
| (Acylglutamate HS21 from the company Ajinomoto) | |
| Liquid petroleum jelly | 0.5% |
| Petroleum jelly | 3% |
| Stearyl heptanoate | 1% |
| Apricot kernel oil | 3% |
| Hydrogenated polyisobutene | 5% |
| Isocetyl palmitate | 5% |
| Volatile silicone | 2% |
| VitaminE | 5% |
| Preserving agent | 0.5% |
| | 0.3% |
| Aqueous phase 1 | |
| Glycerol | 5% |
| Preserving agents | 1% |
| Distilled water | qs 100% |
| Aqueous phase 2 | |
| Carbomer | 0.4% |
| Distilled water | 15% |
| Preserving agents | 0.1% |
| Triethanolamine | 0.4% |
| Aqueous phase 3 | |
| Retinyl propionate nanocapsules according to Example 2, | 10% |
| containing a 2.5% dose of retinyl propionate | |

Procedure: Aqueous phase 1 is introduced at 60° C. into the oily phase, which is also at this temperature, with very vigorous stirring. The temperature and stirring are maintained for 30 minutes. The suspension is then cooled to room temperature. The aqueous phase 2 is then dispersed using a non-shearing disperser. Next, aqueous phase 3 (nanocapsule suspension) is introduced with gentle stirring.

A skincare day cream for combating the signs of ageing is obtained.

The above specification provides a full, clear and concise description of the invention, and the manner and process of making it, such that one of ordinary skill in the art is enabled to make and use the same, this description and enablement being provided in particular for embodiments such as a nanocapsule comprising a lipid core forming or containing at least one lipophilic active agent, and a water-insoluble, preferably continuous, polymer envelope, comprising at least one polyester polyol obtained by polycondensation of at least one aliphatic dicarboxylic acid with at least two alkanediols or with at least one alkanediol and at least one hydroxyalkyl alkanediol optionally comprising an alkyl chain, the polyester polyol having a weight-average molar mass less than 5000, an aqueous suspension of such nanocapsules, a composition comprising an aqueous medium and said nanocapsules, and a composition for topical application, wherein it contains, in a physiologically acceptable support or medium, one or more of such nanocapsules or nanocapsule suspension. Also fully described and enabled is a process for preparing these which comprises:

in dissolving a polymer, a lipid phase forming or containing at least one active agent and optionally a coating agent in a suitable water-miscible organic solvent,

in preparing an aqueous solution of a suitable surfactant in water,

in mixing the organic phase and the aqueous phase, preferably adding the organic phase to the aqueous phase, while stirring,

and then in evaporating the organic phase and, optionally, some of the aqueous phase,

this process being wherein the polymer used in the first step is a polyester polyol as used in the nanocapsule.

Similarly described and enabled is the use of an invention nanocapsule and/or composition thereof as a care, cleansing and/or makeup-removing product for the skin, the hair, the scalp, the eyelashes, the eyebrows, the nails or mucous membranes, or as a makeup product, or as an antisun product, as well as the use of an invention nanocapsule and/or composition thereof for treating a keratin material, especially the skin.

All documents, references, texts, articles, applications, patents, tests, brochures, etc. mentioned herein are incorporated herein by reference.

1. A nanocapsule having a core-envelope structure and comprising:
a lipid core comprising at least one lipophilic active agent, and
a water-insoluble polymer envelope comprising at least one polyester polyol having a weight-average molecular weight less than 5000 obtained by polycondensation of at least one aliphatic dicarboxylic acid with at least two alkanediols or with at least one alkanediol and at least one hydroxyalkyl alkanediol optionally comprising an alkyl chain.

2. The nanocapsule according to claim 1, wherein the aliphatic dicarboxylic acid is selected from the group consisting of malonic acid, succinic acid, glutaric acid, adipic acid, pinclic acid, sebacic acid, azelaic acid, and mixtures thereof.

3. The nanocapsule according to claim 2, wherein the aliphatic dicarboxylic acid is adipic acid.

4. The nanocapsule according to claim 1, wherein the alkanediols are independently selected from the group consisting of alkanediols with a linear or branched chain containing from 2 to 20 carbon atoms.

5. The nanocapsule according to claim 4, wherein the alkanediols are independently selected from the group consisting of ethylene glycol, propylene glycol, 1,3-propanediol, 1,4-butanediol, 1,5-pentanediol, 1,6-hexanediol, neopentyl glycol, and mixtures thereof.

6. The nanocapsule according to claim 1, wherein the hydroxyalkyl alkanediol is selected from the group consisting of 2-alkyl-2-(hydroxyalkyl)-1,3-propanediols, in which the hydroxyalkyl group and the alkyl chain contain, independently of each other, from 1 to 10 carbon atoms; 2-(hydroxyalkyl)-1,3-propanediols, in which the hydroxyalkyl group contains from 1 to 10 carbon atoms; and mixtures thereof, and wherein the polyester polyol having a weight-average molecular weight less than 5000 is obtained by polycondensation of at least one aliphatic dicarboxylic acid with with at least one alkanediol and at least one hydroxyalkyl alkanediol optionally comprising an alkyl chain.

7. The nanocapsule according to claim 1, wherein the polyester polyol is selected from the group consisting of polyester polyols obtained from adipic acid, 1,4-butanediol and 1,6-hexanediol and the polyester polyols obtained from adipic acid, 1,4-butanediol and 2-ethyl-2-(hydroxymethyl)-1,3-propanediol.

8. The nanocapsule according to claim 1, wherein the polyester polyol has a weight-average molecular weight of from 1,000 to 4,500.

9. The nanocapsule according to claim 1, wherein the polyester polyol has a melting point of from 30 to 90° C.

10. The nanocapsule according to claim 1, further comprising an outer lamellar coating on said envelope whose structure is organized as one or more leaflet(s) each comprising a double layer of amphiphilic molecules.

11. The nanocapsule according to claim 10, wherein the said coating comprises a coating agent selected from the group consisting of phospholipids; polycondensates of propylene oxide and of ethylene oxide, silicone surfactants capable of forming lamellar structures; and mixtures thereof.

12. The nanocapsule according to claim 1, wherein the nanocapsules have a mean size of from 50 nm to 800 nm.

13. The nanocapsule according to claim 1, wherein the lipophilic active agent is selected from the group consisting of vitamins and derivatives thereof, emollients, anti-inflammatory agents, antibacterial agents, antifungal agents, antiviral agents, anti-seborrhoeic agents, anti-acne agents, keratolytic agents, antihistamines, anaesthetics, cicatrizing agents, pigmentation modifiers, sunscreens, free-radical traps, moisturizers, and mixtures thereof.

14. The nanocapsule according to claim 1, wherein the lipophilic active agent is selected from the group consisting of retinol and its esters containing from 1 to 30 carbon and mixtures thereof.

15. The nanocapsule according to claim 1, wherein the lipophilic active agent is selected from the group consisting of retinyl propionate, retinyl acetate, retinyl butyrate, retinyl pivalate, retinyl valerate, retinyl hexanoate, retinyl heptanoate, retinyl cyclopentanecarboxylate, retinyl caprate, retinyl caprylate and mixtures thereof.

16. The nanocapsule according to claim 1, wherein the lipophilic active agent is selected from the group consisting of vitamin E or its esters; vitamin D or its derivatives; vitamin F or its derivatives; carotenes; salicylic acid or its derivatives; steroids; and mixtures thereof.

17. The nanocapsule according to claim 1, wherein the polymer envelope is continuous.

18. An aqueous suspension comprising an aqueous medium and, suspended therein, the nanocapsule as claimed in claim 1.

19. A composition comprising an aqueous medium and the nanocapsule as claimed in claim 1.

20. A composition comprising a physiologically acceptable medium and a nanocapsule according to claim 1.

21. The composition according to claim 20, comprising 0.1% to 30% by weight nanocapsules relative to the total weight of the composition.

22. The composition according to claim 21, in the form of a serum, a lotion, an aqueous, aqueous-alcoholic or oily gel, a water-in-oil or oil-in-water emulsion, or an aqueous dispersion of lipid vesicles comprising ionic or nonionic lipids or of a mixture thereof and optionally containing an oily phase.

23. The composition according to claim 20, wherein it constitutes a cosmetic or dermatological composition.

24. A process for preparing the nanocapsule according to claim 1, comprising:
dissolving the polyester polyol, said at least one active agent, and optionally a coating agent, in a water-miscible organic solvent, to prepare an organic phase,
mixing the organic phase with an aqueous phase comprising surfactant, and
evaporating the organic phase and, optionally, some of the aqueous phase.

25. The process according to claim 24, wherein said organic phase is added to said aqueous phase comprising surfactant.

26. A process comprising applying the composition of claim 20 to the skin, the hair, the scalp, the eyelashes, the eyebrows, the nails or mucous membranes.
